(19) United States
(12) Patent Application Publication
(10) Pub. No.: US 2002/0119569 A1
(43) Pub. Date: Aug. 29, 2002

(54) Apparatus for denaturing DEOXYRIBONUCLEIC ACID and fragments thereof

(76) Inventor: John C. Menz, Middleton, WI (US)

Correspondence Address: DAVID J. ARCHER, 9790 FOREST PRESERVE RD., DURAND, IL, 61024, US

(21) Appl. No.: 09/793,194

(22) Filed: Feb. 26, 2001

(51) Int. Cl. C12N 15/01; A61L 2/00; A61L 9/00

(52) U.S. Cl. 435/448; 422/24; 422/299

(57) ABSTRACT

An apparatus is disclosed for denaturing DEOXYRIBONUCLEIC ACID and fragments thereof from laboratory equipment. The apparatus includes a chamber which defines an enclosure. The chamber has an opening for gaining access to the enclosure. A closure is provided for selectively closing the opening. The arrangement is such that when the closure is disposed in a first location thereof relative to the enclosure, the enclosure is sealed and when the closure is disposed in a second location thereof relative to the enclosure, access to the enclosure is permitted. A liner is disposed within the enclosure, the liner having an inside surface, the inside surface of the liner being reflective. An emitter is disposed within the liner, the emitter being selectively connected to a source of electrical power. The arrangement is such that in use of the apparatus, when the emitter is connected to the source of power, ultraviolet radiation is emitted from the emitter. The ultraviolet radiation is progressively reflected within the liner by the reflective inside surface so that when the laboratory equipment is located within the liner, any DEOXYRIBONUCLEIC ACID and fragments thereof present on the equipment is denatured.

### BACKGROUND OF THE INVENTION

1. Field of the Invention

The present invention relates to an apparatus for denaturing DEOXYRIBONUCLEIC ACID (DNA) and fragments thereof from laboratory equipment. More specifically, the present invention relates to an apparatus for denaturing DNA and fragments thereof from laboratory equipment by means of ultraviolet radiation.

2. Background Information

DNA laboratories throughout the world use a chlorine wash decontamination procedure in order to sterilize equipment used in a DNA environment. In such biomedical laboratories, amplified particles of DNA, if not properly contained, can spread unchecked throughout a laboratory environment. DNA particles are known as amplicon and can act as contaminants thus interfering with routine laboratory analyses.

However, the use of a chlorine wash involves considerable technician time and involves the use of harsh chemicals that can adversely affect the laboratory equipment to be sterilized.

The apparatus and method according to the present invention overcomes the aforementioned problems associated with the prior art sterilization arrangements by providing a cost efficient means for decontaminating frequently used equipment. Contaminated laboratory equipment is placed in the apparatus according to the present invention and is exposed to very high input Uvc energy which denatures the DNA particles, rendering such equipment amplicon free. Extensive testing has indicated that the apparatus according to the present invention is effective in removing amplicon contamination without the problems associated with the use of a chlorine wash.

Therefore, it is a primary feature of the present invention to provide an apparatus for denaturing DNA and fragments thereof that overcomes the problems associated with the prior art arrangements.

Another feature of the present invention is the provision of an apparatus for denaturing DNA and fragments thereof and that facilitates removal of such contaminants from laboratory equipment.

A further feature of the present invention is the provision of an apparatus for denaturing DNA and fragments thereof and that reduces the time required by technicians in cleaning laboratory equipment.

Another feature of the present invention is the provision of an apparatus for denaturing DNA and fragments thereof and that avoids the problems associated with use of a chlorine wash for removing contaminants.

Another feature of the present invention is the provision of an apparatus for denaturing DNA and fragments thereof which improves the environmental impact and hygiene of an industrial laboratory.

Another feature of the present invention is the provision of an apparatus for denaturing DNA and fragments thereof which eliminates any environmental problems associated with the use of chlorine or a hypochlorite.

Another feature of the present invention is the provision of an apparatus for denaturing DNA and fragments thereof which overcomes any problems caused by electrical parts becoming exposed to chlorine or a hypochlorite.

Other features and advantages of the present invention will be readily apparent to those skilled in the art by a consideration of the detailed description of a preferred embodiment of the present invention contained herein.

### SUMMARY OF THE INVENTION

The present invention relates to an apparatus for denaturing DEOXYRIBONUCLEIC ACID and fragments thereof from laboratory equipment. The apparatus includes a chamber which defines an enclosure. The chamber has an opening for providing access to the enclosure. A closure is used for selectively closing the opening. The arrangement is such that when the closure is disposed in a first location thereof relative to the enclosure, the enclosure is sealed and when the closure is disposed in a second location thereof relative to the enclosure, access to the enclosure is permitted. A liner is disposed within the enclosure, the liner having an inside surface which is reflective. An emitter is disposed within the liner, the emitter being selectively connected to a source of electrical power. The arrangement is such that in use of the apparatus, when the emitter is connected to the source of power, ultraviolet radiation is emitted from the emitter. The ultraviolet radiation is progressively reflected within the liner by the reflective inside surface so that when the laboratory equipment is located within the liner, any DEOXYRIBONUCLEIC ACID and fragments thereof present on the equipment is denatured.

Throughout the subject specification, it is to be understood by those skilled in the art that the denaturing of DNA and fragments thereof includes denaturing other organic fragments and molecules.

In a more specific embodiment of the present invention, the chamber is fabricated from steel and preferably, the steel of the chamber is stainless steel.

Also, the apparatus further includes casters secured to the chamber for permitting movement thereof.

The chamber is of rectangular box-shaped configuration and includes at least one door which pivots about a vertical axis.

Furthermore, the apparatus includes a lock for locking the door when the closure is disposed in the first location thereof.

An interlocking mechanism is arranged such that when the emitter is connected to the source of electrical power, the interlocking mechanism moves a lock bolt to a first disposition thereof for locking the door. However, when the emitter is disconnected from the source of electrical power, the interlocking mechanism moves the lock bolt to a second disposition thereof for unlocking the door.

Also, the interlocking mechanism has a solenoid which cooperates with the lock bolt so that the solenoid is energized when the emitter is electrically connected to the source of electrical power. The mechanism is arranged such that the lock locks the door so that access to the enclosure is prevented.

The inside surface of the liner has at least four corners for assisting in focusing the ultraviolet radiation towards the laboratory equipment within the liner.

Moreover, the liner is fabricated from aluminum and the inside surface of the aluminum liner is polished to enhance reflection of the radiation.

The emitter includes a first UV emitter and a second UV emitter disposed angularly relative to the first UV emitter for maximizing bombardment of the equipment by photons emitted by the UV emitters so that denaturing of the DEOXYRIBONUCLEIC ACID and fragments thereof is accomplished rapidly.

Also, a Radiometer is provided within the liner for measuring UV radiation so that the life of the emitter is maximized and so that radiation from the emitter is monitored.

The present invention also includes a method for denaturing DEOXYRIBONUCLEIC ACID and fragments thereof from laboratory equipment. The method includes the steps of providing a chamber which defines an enclosure, the chamber having an opening which provides access to the enclosure.

The method also includes selectively closing the opening with a closure, the arrangement being such that when the closure is disposed in a first location thereof relative to the enclosure, the enclosure is sealed and when the closure is disposed in a second location thereof relative to the enclosure, access to the enclosure is permitted.

The laboratory equipment is then placed within a liner disposed within the enclosure, the liner defining an inside surface, the inside surface of the liner being reflective.

Finally, an emitter disposed within the liner is connected to a source of electrical power such that when the emitter is connected to the source of power, ultraviolet radiation is emitted from the emitter. The ultraviolet radiation is progressively reflected within the liner by the reflective inside surface so that any DEOXYRIBONUCLEIC ACID and fragments thereof present on the equipment is denatured.

The radiation is high power radiation such that a high flux density is achieved within the liner. The high flux is achieved by focusing reflectance as the radiation is progressively reflected particularly by means of three converging planes defined at each of the eight corners of the liner which is made up of six sides.

Many modifications and variations of the present invention will be readily apparent to those skilled in the art by a consideration of the detailed description contained hereinafter taken in conjunction with the annexed drawings which show a preferred embodiment of the present invention. However, such modifications and variations fall within the spirit and scope of the present invention as defined by the appended claims.

Included amongst such modifications would be the provision of an apparatus in which the liner is formed as a one piece unit with the chamber rather than as separate liner.

### BRIEF DESCRIPTION OF THE DRAWINGS

FIG. 1 is a perspective view of an apparatus according to the present invention for denaturing DEOXYRIBONUCLEIC ACID and fragments thereof from laboratory equipment;

FIG. 2 is a front elevational view of the apparatus shown in FIG. 1;

FIG. 3 is a perspective view of a liner removed from an enclosure of the apparatus shown in FIG. 1;

FIG. 4 is a front elevational view similar to that shown in FIG. 2 but with a door of the apparatus in a closed disposition thereof;

FIG. 5 is a schematic of the electrical wiring of the apparatus shown in FIG. 1;

FIG. 6 is a right hand side elevational view of the apparatus shown in FIG. 1;

FIG. 7 is a perspective view of a preferred embodiment of the apparatus according to the present invention;

FIG. 8 is a left hand elevational view of the apparatus shown in FIG. 7;

FIG. 9 is a top plan view of the apparatus shown in FIG. 7;

FIG. 10 is a front elevational view of the apparatus shown in FIG. 7; and

FIG. 11 is a similar view to that shown in FIG. 10 but with the closure opened.

Similar reference characters refer to similar parts throughout the various views of the drawings.

### DETAILED DESCRIPTION OF THE DRAWINGS

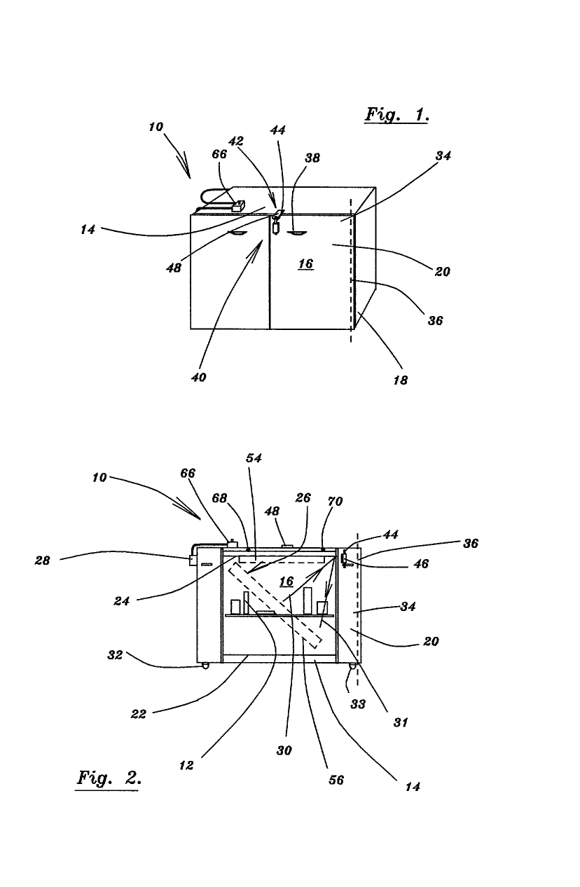

FIG. 1 is a perspective view of an apparatus generally designated 10 according to the present invention for denaturing DEOXYRIBONUCLEIC ACID and fragments thereof from laboratory equipment. The apparatus 10 includes a chamber 14 which defines an enclosure 16. The chamber 14 has an opening 18 for providing access to the enclosure 16. A closure 20 is provided for selectively closing the opening 18. The arrangement is such that when the closure 20 is disposed in a first location thereof relative to the enclosure 16 as shown in FIG. 1, the enclosure 16 is sealed.

FIG. 2 is a front elevational view of the apparatus 10 shown in FIG. 1. However, FIG. 2 shows the closure 20 disposed in a second location thereof relative to the enclosure 16, such that access to the enclosure 16 is permitted. A liner 22 is disposed within the enclosure 16, the liner 22 having an inside surface 24. Moreover, the inside surface 24 of the liner 22 is reflective. An emitter generally designated 26 and shown in phantom outline, is disposed within the liner 22. The emitter 26 is selectively connected to a source of electrical power 28. The arrangement is such that in use of the apparatus 10, when the emitter 26 is connected to the source of power 28, ultraviolet radiation as indicated by the arrows 30 and 31 is emitted from the emitter 26. The ultraviolet radiation 30-31 is progressively reflected within the liner 22 by the reflective inside surface 24 of the liner 22 so that when laboratory equipment 12 is located within the liner 22, any DEOXYRIBONUCLEIC ACID and fragments thereof present on the laboratory equipment 12 is denatured.

In a more specific embodiment of the present invention, the chamber 14 is fabricated from steel and preferably, the steel of the chamber 14 is stainless steel.

As shown in FIG. 2, the apparatus 10 also includes four casters of which casters 32 and 33 are shown secured beneath the chamber 14 for permitting movement thereof.

The chamber 14 is of rectangular box-shaped configuration as shown in FIG. 1 and includes at least one door 34 which pivots about a vertical axis 36. The door 34 includes a handle 38 for opening the door 34.

Furthermore, the apparatus 10 includes a lock generally designated 40 for locking the door 34 when the closure 20 is disposed in the first location thereof as shown in FIG. 1.

An interlocking mechanism generally designated 42 is arranged such that when the emitter 26 is connected to the source of electrical power 28, the interlocking mechanism 42 moves a lock bolt 44 to a first disposition thereof for locking the door 34 as shown in FIG. 1. However, when the emitter 26 is disconnected from the source of electrical power 28, the interlocking mechanism 42 moves the lock bolt 44 to a second disposition thereof for unlocking the door 34 as shown in FIG. 2.

Also, the interlocking mechanism 42 has a solenoid 46 which cooperates with the lock bolt 44 so that the solenoid 46 is energized when the emitter 26 is electrically connected to the source of electrical power 28. The arrangement is such that the lock bolt 44 engages a catch 48 for locking the door 34 so that access to the enclosure 16 during the denaturing process is prevented.

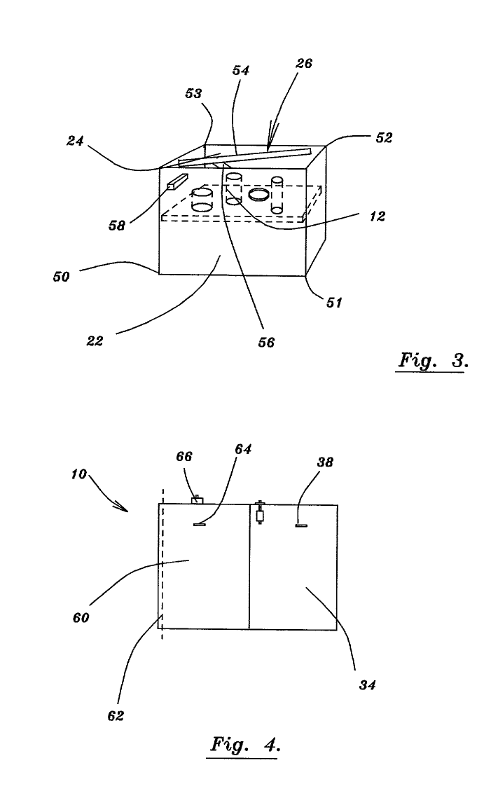

FIG. 3 is a perspective view of the liner 22 removed from the enclosure 16. As shown in FIG. 3, the inside surface 24 of the liner 22 has at least four corners 50, 51, 52 and 53 for assisting in focusing the ultraviolet radiation 30-31 towards the laboratory equipment 12 within the liner 22.

Moreover, the liner 22 is fabricated from aluminum and the inside surface 24 of the aluminum liner 22 is polished to enhance reflection of the radiation 30-31.

As shown in FIG. 3, the emitter 26 includes a first UV emitter 54 and a second UV emitter 56 disposed angularly relative to the first UV emitter 54 for maximizing bombardment of the equipment 12 by photons emitted by the UV emitters 54 and 56 so that denaturing of the DEOXYRIBONUCLEIC ACID and fragments thereof is accomplished rapidly.

Also, a Radiometer 58 is provided within the liner 22 for measuring UV radiation 30-31 so that the life of the emitter 26 is maximized and so that radiation 30-31 from the emitter 26 is monitored.

FIG. 4 is a front elevational view similar to that shown in FIG. 2 showing the apparatus 10 but with the door 34 in the first closed disposition thereof. As shown in FIG. 4, a further door 60 is provided. The further door 60 hinges about a further pivotal axis 62. Also, the further door 60 is provided with a further handle 64 for permitting opening of the further door 60.

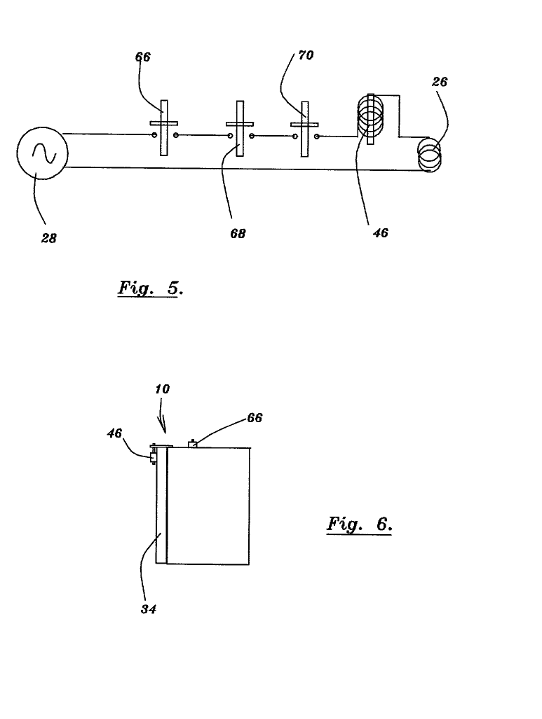

FIG. 5 is a schematic of the electrical wiring of the apparatus 10. As shown in FIG. 5, an on/off switch 66 is electrically connected between the source of electrical power 28 and the emitter 26. The emitter 26 is also electrically connected to the solenoid 46 so that when the switch 66 is turned on, the lock 40 locks the door 34 in the first closed disposition thereof. Contact switches 68 and 70 also shown in FIG. 2 are electrically connected between the on/off switch 66 and the solenoid 46 so that the emitter radiates only when the doors 34 and 60 are closed and when the solenoid 46 is energized for locking the doors.

FIG. 6 is a right hand side elevational view of the apparatus 10 showing the door 34, solenoid 46 and the on/off switch 66.

In operation of the apparatus 10 shown in FIGS. 1-6, the laboratory equipment 12 which has been used during DNA procedures is placed within the liner 22 and the doors 34 and 60 are closed. The switch 66 is turned on. Accordingly, the doors 34 and 60 are locked by an interaction between the lock bolt 44 and the catch 48. Also, the emitters 54 and 56 emit a high output of UVc energy which in conjunction with the polished reflective inside surface 24 of the liner 22 produces a very high flux density photon bombardment which ensures the complete denaturization of the target DNA. The fully enclosed and polished aluminum interior of the liner 22 reflects the UVc photons until they hit the target equipment 12 and are absorbed. The corners 50-53 of the rectangular shaped liner 22 help focus reflected photons towards the target equipment 12. Also, the global emission of photons from the bulb of the emitters 54 and 56 ensure photon bombardment of the target equipment 12 from every angle.

The time that the equipment is exposed to radiation depends on the requirements of the user of the apparatus. However, tests have indicated that a 2 minute exposure on all sides of the target equipment 12 is effective for 100% denaturization of DNA fragments.

Although, in the aforementioned embodiment of the present invention, the apparatus is of stand-alone, refrigerator style configuration, the concept of the present invention is equally applicable to a wall mounted unit or the like. Also, the present invention envisages a very high output UV emitter and a cylindrical, fully enclosed system for long, thin moisture probes that cannot be wetted due to moisture calibration.

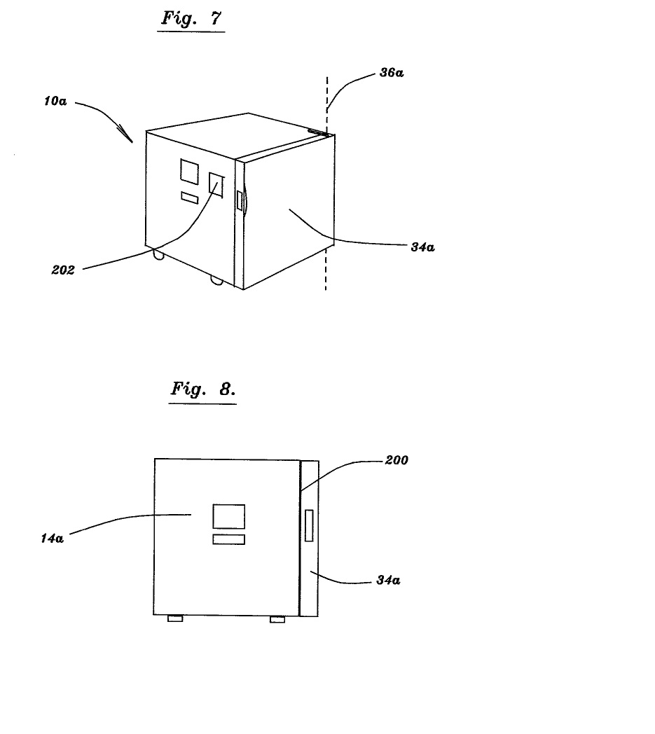

FIG. 7 is a perspective view of a preferred embodiment of the apparatus according to the present invention. As shown in FIG. 7, the apparatus 10a includes a single door 34a which hinges about pivotal axis 36a.

FIG. 8 is a left hand elevational view of the apparatus shown in FIG. 7. As shown in FIG. 8, a magnetic strip 200 is disposed between the door 34a and the chamber 14a so that when the door is closed, the magnetic strip 200 will seal the door 34a against the chamber 14a.

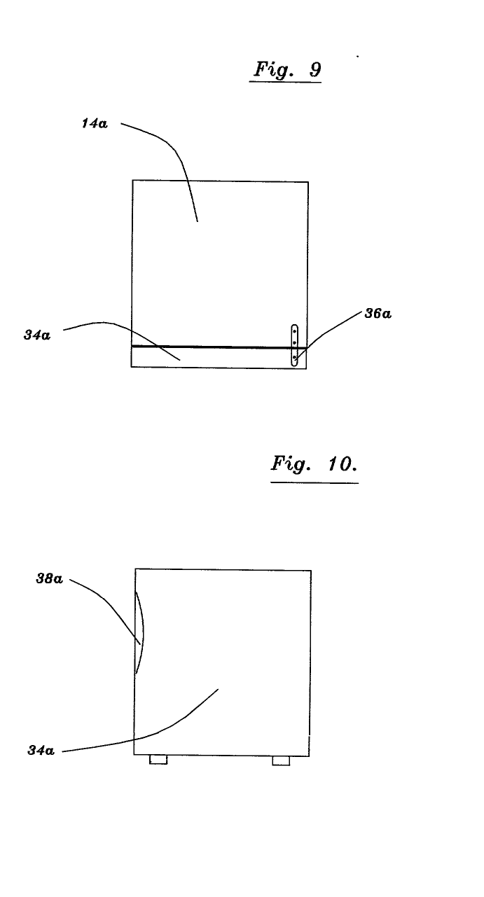

FIG. 9 is a top plan view of the apparatus shown in FIG. 7. As shown in FIG. 9, the door 34a hinges about pivotal axis 36a to the closed disposition against the chamber 14a.

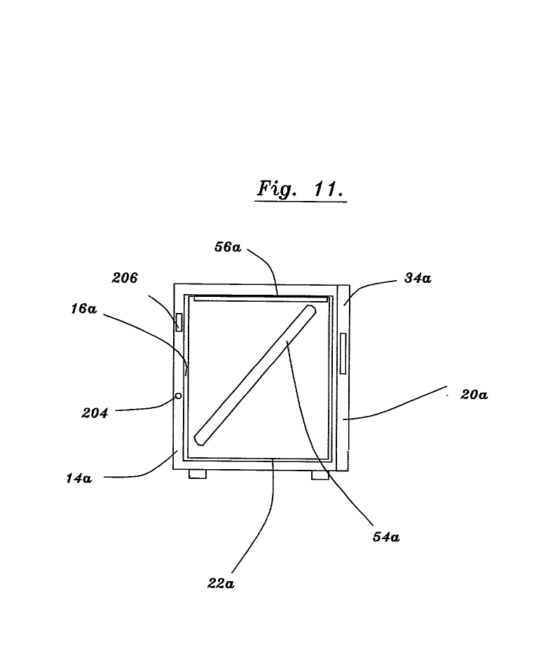

FIG. 10 is a front elevational view of the apparatus shown in FIG. 7. As shown in FIG. 10, the door 34a includes a shaped handle 38a.

FIG. 11 is a similar view to that shown in FIG. 10 but with the closure 20a opened. As shown in FIG. 11, the closure 20a which is a door 34a is opened to show the enclosure 16a defined by the chamber 14a. Also, a liner 22a is shown within the enclosure 16a with a first and second UV emitter 54a and 56a respectively secured to the liner 22a.

Additionally, as shown in FIG. 7, in operation of the apparatus 10a as shown in FIGS. 7-11, a timer control 202 is provided such that when the timer 202 is set and the door 34a is closed, such closing of the door 34a closes a switch 204 of an electrical circuit, as shown in FIG. 11, so that the emitters 54a and 56a are energized. Also, as shown in FIG. 11, a magnetic strip 206 releasably holds the door 34 in the closed disposition thereof..

The present invention provides a unique apparatus for denaturing DNA and fragments thereof using UV radiation.

What is claimed is:

1. An apparatus for denaturing DEOXYRIBONUCLEIC ACID and fragments thereof from laboratory equipment, said apparatus comprising:
a chamber defining an enclosure;
said chamber defining an opening for providing access to said enclosure;
a closure for selectively closing said opening, the arrangement being such that when said closure is disposed in a first location thereof relative to said enclosure, said enclosure is sealed and when said closure is disposed in a second location thereof relative to said enclosure, access to said enclosure is permitted;
a liner disposed within said enclosure, said liner defining an inside surface;
said inside surface of said liner being reflective; and
an emitter disposed within said liner, said emitter being selectively connected to a source of electrical power such that in use of the apparatus, when said emitter is connected to said source of power, ultraviolet radiation is emitted from said emitter, said ultraviolet radiation being progressively reflected within said liner by said reflective inside surface so that when the laboratory equipment is located within said liner, any DEOXYRIBONUCLEIC ACID and fragments thereof present on the equipment is denatured.

2. An apparatus for denaturing DEOXYRIBONUCLEIC ACID and fragments thereof as set forth in claim 1 wherein
said chamber is fabricated from steel.

3. An apparatus for denaturing DEOXYRIBONUCLEIC ACID and fragments thereof as set forth in claim 2 wherein
said steel of said chamber is stainless steel.

4. An apparatus for denaturing DEOXYRIBONUCLEIC ACID and fragments thereof as set forth in claim 1 further including:
a plurality of casters secured to said chamber for permitting movement thereof.

5. An apparatus for denaturing DEOXYRIBONUCLEIC ACID and fragments thereof as set forth in claim 1 wherein
said chamber is of rectangular box-shaped configuration.

6. An apparatus for denaturing DEOXYRIBONUCLEIC ACID and fragments thereof as set forth in claim 1 wherein
said closure includes:
at least one door.

7. An apparatus for denaturing DEOXYRIBONUCLEIC ACID and fragments thereof as set forth in claim 1 wherein
said at least one door pivots about a vertical axis.

8. An apparatus for denaturing DEOXYRIBONUCLEIC ACID and fragments thereof as set forth in claim 7 further including:
a lock bolt for locking said at least one door when said closure is disposed in said first location thereof.

9. An apparatus for denaturing DEOXYRIBONUCLEIC ACID and fragments thereof as set forth in claim 8 further including:
an interlocking mechanism arranged such that when said emitter is connected to the source of electrical power, said interlocking mechanism moves said lock bolt to a first disposition thereof for locking said at least one door and when said emitter is disconnected from the source of electrical power, said interlocking mechanism moves said lock bolt to a second disposition thereof for unlocking said at least one door.

10. An apparatus for denaturing DEOXYRIBONUCLEIC ACID and fragments thereof as set forth in claim 9 wherein
said interlocking mechanism further includes:
a solenoid cooperating with said lock bolt so that said solenoid is energized when said emitter is electrically connected to the source of electrical power such that said lock bolt locks said at least one door so that access to said enclosure is prevented.

11. An apparatus for denaturing DEOXYRIBONUCLEIC ACID and fragments thereof as set forth in claim 1 wherein
said inside surface of said liner defines at least four corners for assisting in focusing said ultraviolet radiation towards the laboratory equipment within said liner.

12. An apparatus for denaturing DEOXYRIBONUCLEIC ACID and fragments thereof as set forth in claim 1 wherein
said liner is fabricated from aluminum.

13. An apparatus for denaturing DEOXYRIBONUCLEIC ACID and fragments thereof as set forth in claim 12 wherein
said inside surface of said aluminum liner is polished to enhance reflection of said radiation.

14. An apparatus for denaturing DEOXYRIBONUCLEIC ACID and fragments thereof as set forth in claim 1 wherein
said emitter includes:
a first UV emitter;
a second UV emitter disposed angularly relative to said first UV emitter for maximizing bombardment of the equipment by photons emitted by said UV emitters so that denaturing of the DEOXYRIBONUCLEIC ACID and fragments thereof is accomplished rapidly.

15. An apparatus for denaturing DEOXYRIBONUCLEIC ACID and fragments thereof as set forth in claim 1 further including:
a Radiometer disposed within said liner for measuring UV radiation so that the life of said emitter is maximized and so that radiation from said emitter is monitored.

16. An apparatus for denaturing DEOXYRIBONUCLEIC ACID and fragments thereof from laboratory equipment, said apparatus comprising:
a chamber defining an enclosure;
said chamber defining an opening for providing access to said enclosure;
a closure for selectively closing said opening, the arrangement being such that when said closure is disposed in a first location thereof relative to said enclosure, said enclosure is sealed and when said closure is disposed in a second location thereof relative to said enclosure, access to said enclosure is permitted;
a six-sided liner disposed within said enclosure, said liner defining an inside surface;
said inside surface of said liner being reflective; and
an emitter disposed within said liner, said emitter being selectively connected to a source of electrical power such that in use of the apparatus, when said emitter is connected to said source of power, ultraviolet radiation is emitted from said emitter, said ultraviolet radiation being progressively reflected within said liner by said reflective inside surface so that when the laboratory equipment is located within said liner, any DEOXYRIBONUCLEIC ACID and fragments thereof present on the equipment is denatured.

17. An apparatus for denaturing DEOXYRIBONUCLEIC ACID and fragments thereof as set forth in claim 16 wherein
said radiation is high power radiation such that a high flux density is achieved within said liner, said high flux being achieved by focusing said reflected radiation as said radiation is progressively reflected particularly by means of three converging planes defined at each corner of said six-sided liner.

18. A method for denaturing DEOXYRIBONUCLEIC ACID and fragments thereof from laboratory equipment, the method comprising the steps of:
providing a chamber which defines an enclosure, the chamber having an opening which permits access to the enclosure;
selectively closing the opening with a closure, the arrangement being such that when the closure is disposed in a first location thereof relative to the enclosure, the enclosure is sealed and when the closure is disposed in a second location thereof relative to the enclosure, access to the enclosure is permitted;
placing the laboratory equipment within a liner disposed within the enclosure, the liner defining an inside surface, the inside surface of the liner being reflective; and
connecting an emitter disposed within the liner to a source of electrical power such that when the emitter is connected to the source of power, ultraviolet radiation is emitted from the emitter, the ultraviolet radiation being progressively reflected within the liner by the reflective inside surface so that any DEOXYRIBONUCLEIC ACID and fragments thereof present on the equipment is denatured.
